(19) United States
(12) Patent Application Publication
(10) Pub. No.: US 2008/0021378 A1
(43) Pub. Date: Jan. 24, 2008

(54) COLON HYDROTHERAPY APPARATUS

(76) Inventor: Gary Johnson, Buckeye, AZ (US)

Correspondence Address: SNELL & WILMER L.L.P. (Main), 400 EAST VAN BUREN, ONE ARIZONA CENTER, PHOENIX, AZ, 85004-2202, US

(21) Appl. No.: 11/761,296

(22) Filed: Jun. 11, 2007

Related U.S. Application Data
(60) Provisional application No. 60/812,663, filed on Jun. 9, 2006.

(51) Int. Cl. A61M 3/02

(52) U.S. Cl. 604/30

(57) ABSTRACT

A colon hydrotherapy apparatus having first and second modular units connected to one another where fluid flow components are contained in the first modular unit and electronic components which operate and control the fluid flow components are contained in the second modular unit. Pressure and temperature manifolds comprised of a polymer material are also included in the colon hydrotherapy apparatus.

### CROSS-REFERENCE TO RELATED APPLICATIONS

This application claims priority to provisional patent application having Ser. No. 60/812,663, filed Jun. 9, 2006, which is herein incorporated in its entirety.

### FIELD OF INVENTION

The present invention is generally related to a colon hydrotherapy apparatus and method for making same. More particularly, the present invention is related to a colon hydrotherapy apparatus which utilizes plastic or polymer manifolds to increase the accuracy of the water temperature and pressure that is employed during use of the apparatus.

### BACKGROUND OF THE INVENTION

Numerous types of systems and apparatus for irrigating and cleansing the colon can be found in the prior art. Prior art systems and apparatus typically include a fluid (such as water) inlet for introducing water into the apparatus, means for purifying the water such as a filter system, means for controlling the fluid flow and fluid pressure, and a fluid outlet which is in turn connected to a rectal speculum via a tubing. The prior art systems and apparatus for colon irrigation/hydrotherapy include temperature and flow components which are controlled by electronic components, all of which are contained within the same housing or compartment.

Enclosing the temperature and fluid flow components and electronic components within the same housing increases the potential for the apparatus to malfunction in the event of a fluid leak and also creates hazards with respect to safety. In addition, the temperature and pressure manifolds used in such systems and apparatus are comprised of brass or metal which are capable of corroding and acting as heat sinks. Furthermore, manifolds made of brass or metal may pose problems with potential toxicity levels.

Accordingly, there is a need for a colon hydrotherapy system and apparatus which reduces the potential for malfunction of the apparatus in addition to reducing safety hazards. There is also a need for a colon hydrotherapy system and apparatus which utilizes plastic or polymer manifolds thereby creating a number of advantages over prior art systems and apparatus.

### SUMMARY OF THE INVENTION

The present invention is directed to a colon hydrotherapy apparatus which includes a first modular unit which contains fluid flow components and a second modular unit connected to the first modular unit where the second modular unit contains electronic components for operating the fluid flow components. The fluid flow components contained in the first modular unit include a pressure manifold and a temperature manifold.

The present invention is also directed to a pressure manifold and a temperature manifold which are each comprised of a polymer or plastic type of material. In one exemplary embodiment of the present invention, the pressure manifold includes a polymer block member having a first circular opening and a second circular opening contained within a first side of the polymer block member, a first tunnel and a second tunnel contiguous with the first and second circular openings wherein the circumference of the first and second tunnels is smaller than the circumference of the first and second circular openings, a third circular opening within a second side of the polymer block member, and a third tunnel contiguous with the third circular opening where the circumference of the third tunnel is equal to a circumference of the third circular opening and the first and second hollow tunnels are contiguous with, and open into, the third hollow tunnel. In one aspect of this exemplary embodiment of the invention, the third tunnel may be larger in circumference than the first and second tunnels. In another aspect of this exemplary embodiment of the invention, the third circular opening may have a circumference equal to or larger than the circumference of the first and second circular openings.

Another exemplary embodiment of the present invention includes a temperature manifold made of a polymer block member which includes a first circular opening and a second circular opening within a first side of the polymer block member, a first tunnel and a second tunnel contiguous with the first and second circular openings where the circumference of the first and second tunnels is smaller than the circumference of the first and second circular openings, a third circular opening within the second side of the polymer block member, and a third tunnel contiguous with the third circular opening where a circumference of the third tunnel is smaller than a circumference of the third circular opening and the first and second hollow tunnels are contiguous with and open into the third hollow tunnel. In one aspect of this exemplary embodiment of the invention, the third tunnel may be equal in circumference to the circumference of the first and second tunnels. In another aspect of this exemplary embodiment of the invention, the third circular opening may have a circumference equal to or larger than a circumference of the first and second circular openings.

An exemplary embodiment of the colon hydrotherapy apparatus of the present invention may also include a mixing valve and a timer within the first modular unit in addition to the pressure manifold and the temperature manifold.

### BRIEF DESCRIPTION OF THE DRAWINGS

The present invention will hereinafter be described in conjunction with the appended drawing figures where like numerals denote like elements, and:

FIG. 1 is a front elevational view of an exemplary colon hydrotherapy apparatus of the present invention showing the first and second modular units;

FIG. 2 is a perspective view of an exemplary embodiment of the colon hydrotherapy apparatus of the present invention showing the modular unit which contains the fluid flow components including the pressure and temperature manifolds;

FIG. 3 is a perspective view of an exemplary embodiment of the pressure manifold contained in the colon hydrotherapy apparatus of the present invention;

FIG. 4 is a schematic showing the tooling for the pressure manifold shown in FIG. 3;

FIG. 5 is a perspective view of an exemplary embodiment of the temperature manifold contained in the colon hydrotherapy apparatus of the present invention;

FIG. 6 is a schematic showing the tooling for the temperature manifold shown in FIG. 5.

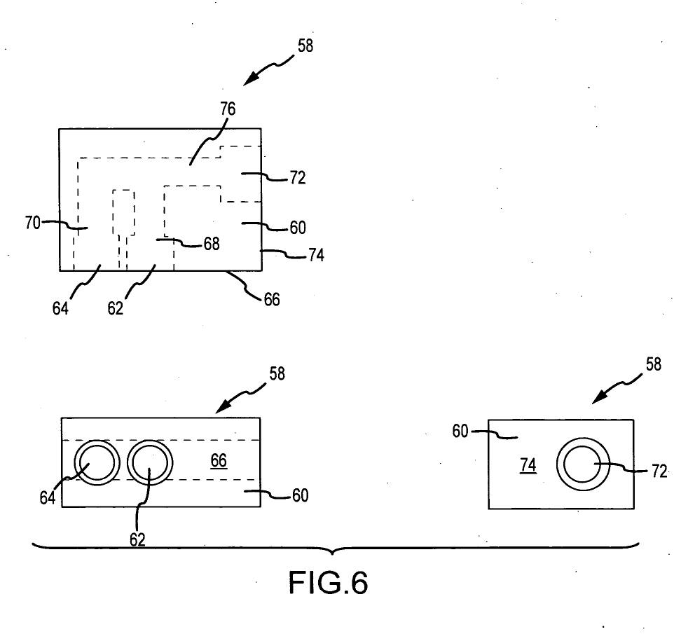

### DETAILED DESCRIPTION

The colon hydrotherapy apparatus of the present invention generally includes a first modular unit having fluid flow components contained therein and a second modular unit connected to the first modular unit where the second modular unit includes electronic components which operate the fluid flow components. The colon hydrotherapy apparatus also includes at least a pressure manifold and a temperature manifold which are preferably comprised of a polymer or like material. These manifolds also may comprise particular configurations which create a number of advantages over prior art colon cleansing systems and apparatus.

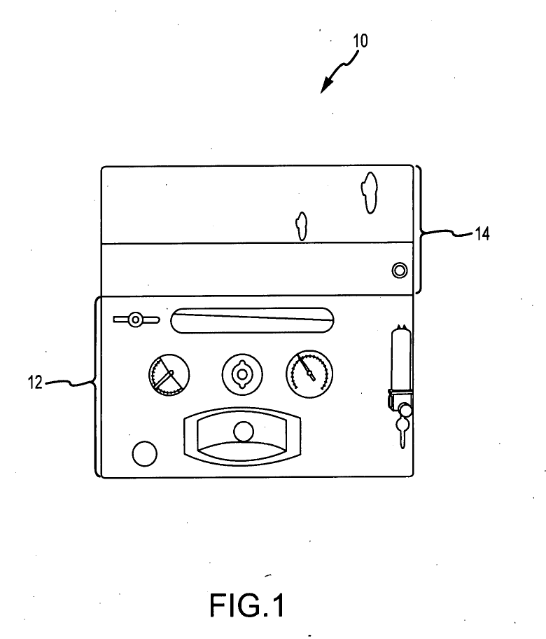

FIG. 1 shows a front elevational view of an exemplary embodiment of the colon hydrotherapy apparatus 10 of the present invention. The colon hydrotherapy apparatus 10 includes a first modular unit 12 connected to a second modular unit 14. The first modular unit 12 includes fluid flow components while the second modular unit contains electronic components which operate the fluid flow components contained in the first modular unit 12.

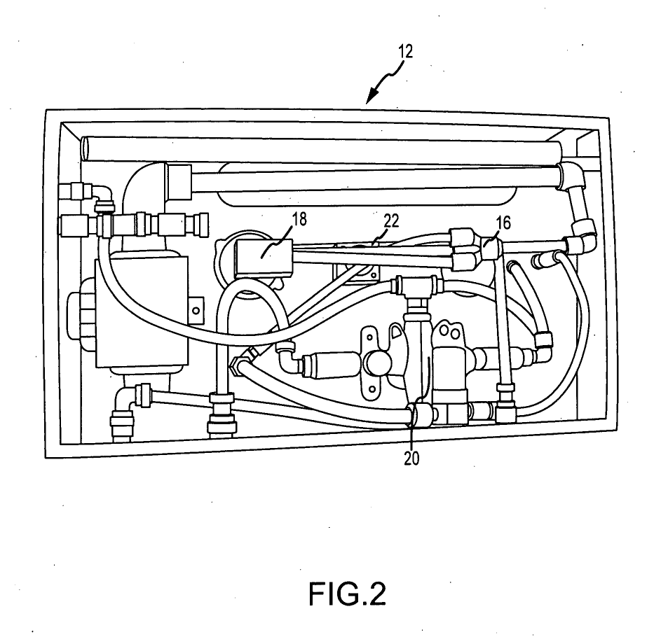

A perspective view of an exemplary embodiment of the first modular unit 12 of the colon hydrotherapy apparatus of the present invention is shown in FIG. 2. The perspective view depicted in FIG. 2 shows the first modular unit 12 with its back panel removed so that the fluid flow components contained within first modular unit 12 can be seen. The fluid flow components in the first modular unit 12 include a pressure manifold 16 and a temperature manifold 18. Fluid flow components contained in first modular unit 12 may also include a mixing valve 20 and a timer 22. As can be seen in FIG. 2, the fluid flow components contained in first modular unit 12 are completely separate from the electronic components which control the fluid flow components. The electronic components are contained in the second modular unit 14 which is placed on top of, and connected to, first modular unit 12. Separating the fluid flow components from the electronic components which operate and control the fluid flow components reduces the likelihood of a malfunctioning apparatus in the event of a fluid leak which, in prior art apparatus, would come into contact with the electronic components. Separating the fluid flow components from the electronic components also reduces safety hazards and further ensures that one operating the apparatus will not be injured in the event that a fluid leak occurs.

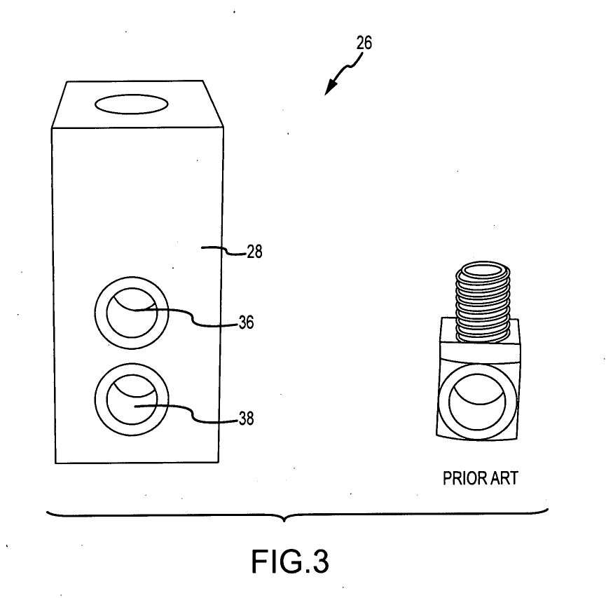

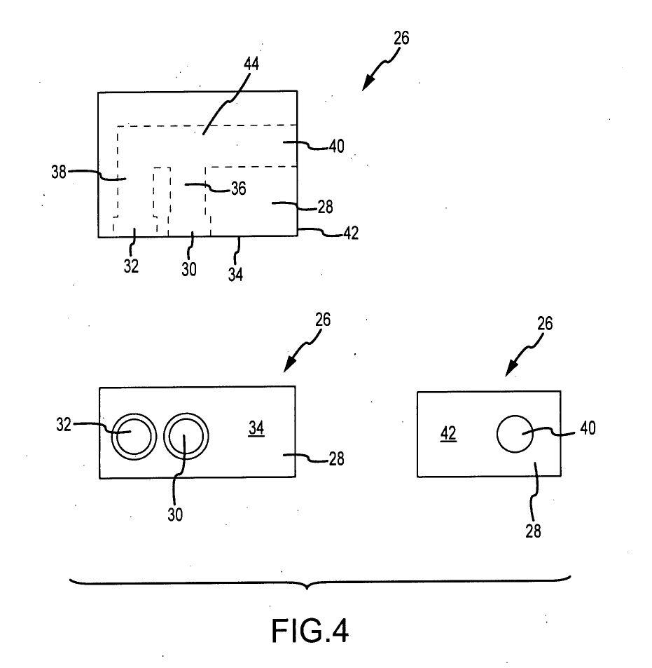

FIG. 3 is a perspective view of an exemplary embodiment of the pressure manifold contained in the colon hydrotherapy apparatus of the present invention. A perspective view of a prior art manifold is also shown in FIG. 3. A schematic showing the tooling for the pressure manifold in FIG. 3 is depicted in FIG. 4. Pressure manifold 26 includes a polymer block member 28 having a first circular opening 30 and a second circular opening 32 within a first side 34 of polymer block member 28 and a first tunnel 36 and a second tunnel 38 contiguous with the first and second circular openings 30, 32, respectively, where a circumference of the first and second tunnels 36, 38 is smaller than a circumference of the first and second circular openings 30, 32. Polymer block member 28 also includes a third circular opening 40 within a second side 42 of polymer block member 28 and a third tunnel 44 contiguous with a third circular opening 40 where a circumference of the third tunnel 44 is equal to the circumference of the third circular opening 40 and the first and second hollow tunnels 36, 38 are contiguous with, and open into, the third hollow tunnel 44. The third tunnel 44 may be larger in circumference than first and second tunnels 36, 38. In addition, third circular opening 40 may have a circumference equal to or larger than a circumference of the first and second circular openings 30, 32.

Pressure manifold 26 is preferably comprised of a polymer block member 28 which may include any type of polymer or like material having the same characteristics and properties of polymer or plastic. Some examples include, but are not limited to, polyethylene oxide, polyethyleneterephthalate, polytetrafluoroethylene, and nylon. The pressure manifold of the present invention shown in FIGS. 3 and 4 has many advantages over prior art manifolds or fittings which are used to carry out the same function as a fluid flow component in a colon hydrotherapy system, device or apparatus. One advantage of the pressure manifold 26 of the present invention is that there is no potential for brass or metal toxicity levels in that the pressure manifold is comprised of a polymer or plastic material and not of brass or metal. Further, the plastic or polymer pressure manifold of the present invention will not corrode and does not act as a heat sink like prior art manifolds. The pressure manifold of the present invention also has the following advantages: 1) it reduces pressure gage response times, 2) it reduces the amount of materials needed such as poly-tubing and brass materials, 3) it reduces assembly and repair man hours, 4) it balances internal pressure, 5) it eliminates dead-end connections, 6) it eliminates trapped air, 7) it eliminates T-nut torque specifications required for brass and metal fittings, 8) it reduces residual water contained in the apparatus when a full system shut down and drain procedure is required, and 9) it reduces overall weight of the apparatus.

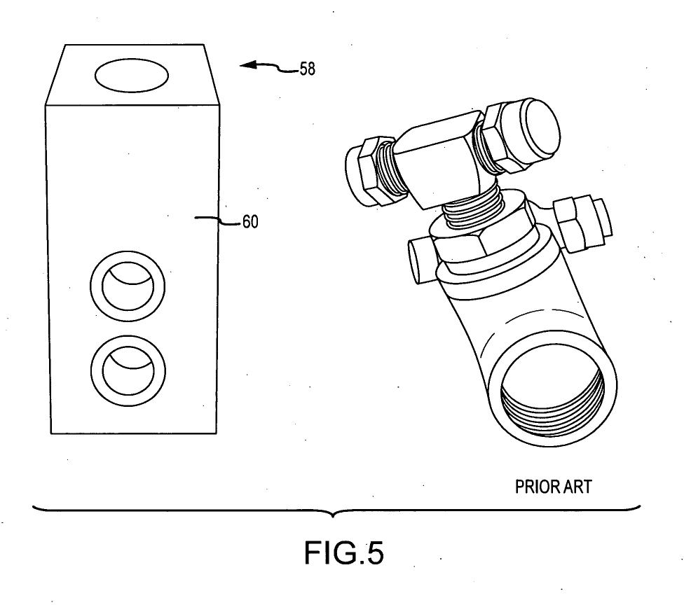

The present invention is also directed to a temperature manifold for use in a colon hydrotherapy apparatus or system. FIG. 4 is a perspective view of an exemplary embodiment of a temperature manifold 58 contained in the colon hydrotherapy apparatus of the present invention. A perspective view of a prior art temperature manifold is also shown in FIG. 5. A schematic showing the tooling for the temperature manifold shown in FIG. 4 is depicted in FIG. 5. Temperature manifold 58 of the present invention comprises a polymer block member 60 which includes a first circular opening 62 and a second circular opening 64 within a first side 66 of the polymer block member 58 and a first tunnel 68 and a second tunnel 70 contiguous with the first and second circular openings 62, 64, respectively, where the circumference of the first and second tunnels 68, 70 is smaller than the circumference of the first and second circular openings 62, 64. Polymer block member 60 also includes a third circular opening 72 within a second side 74 of polymer block member 60 and a third tunnel 76 contiguous with the third circular opening 72 where the circumference of the third tunnel 76 is smaller than a circumference of the third circular opening 72 and the first and second hollow tunnels 68, 70 are contiguous with, and open into, the third hollow tunnel 76. The third tunnel 76 may be equal in circumference to the circumference of the first and second tunnels 68, 70. In addition, the third circular opening 72 may have a circumference equal to or larger than a circumference of the first and second circular openings 62, 64.

The temperature manifold 58 of the present invention which is comprised of polymer or a similar material having the same properties or characteristics has the same advantages as pressure manifold 26 of the present invention. One advantage of the temperature manifold 58 of the present invention is that there is no potential for brass or metal toxicity levels in that the temperature manifold is comprised of a polymer or plastic material and not of brass or metal. Further, the plastic or polymer temperature manifold of the present invention will not corrode and does not act as a heat sink like prior art manifolds. The temperature manifold of the present invention also has the following advantages: 1) it reduces temperature gage response times, 2) it reduces the amount of materials needed such as poly-tubing and brass materials, 3) it reduces assembly and repair man hours, 4) it balances internal temperature, 5) it eliminates dead-end connections, 6) it eliminates trapped air, 7) it eliminates T-nut torque specifications required for brass and metal fittings, 8) it reduces residual water contained in the apparatus when a full system shut down and drain procedure is required, and 9) it reduces overall weight of the apparatus.

Many modifications and other embodiments of the invention set forth herein will come to mind to one skilled in the art to which this invention pertains having the benefit of the teachings presented in the foregoing descriptions and the associated drawings. Therefore, it is to be understood that the invention is not to be limited to the specific embodiments disclosed and that modifications and other embodiments are intended to be included within the scope of the appended claims. Although specific terms are employed herein, they are used in a generic and descriptive sense only and not for purposes of limitation.

1. A colon hydrotherapy apparatus comprising:
A first modular unit having fluid flow components contained therein; and
A second modular unit connected to the first modular unit, said second modular unit having electronic components contained therein for operating said fluid flow components.

2. The colon hydrotherapy apparatus of claim 1 wherein said fluid flow components include at least a pressure manifold and a temperature manifold.

3. The colon hydrotherapy apparatus of claim 2 wherein said pressure manifold and said temperature manifold are each comprised of a polymer.

4. The colon hydrotherapy device of claim 2 wherein said fluid flow components further comprise a mixing valve.

5. The colon hydrotherapy apparatus of claim 2 wherein said first modular unit further includes a timer.

6. The colon hydrotherapy device of claim 2 wherein said pressure manifold comprises a polymer block member having:
a first circular opening and a second circular opening within a first side of said polymer block member;
a first tunnel and a second tunnel contiguous with said first and second circular openings, respectively, wherein a circumference of said first and second tunnels is smaller than a circumference of said first and second circular openings;
a third circular opening within a second side of said polymer block member; and
a third tunnel contiguous with the third circular opening wherein a circumference of said third tunnel is equal to a circumference of said third circular opening and said first and second tunnels are contiguous with and open into said third tunnel.

7. The colon hydrotherapy apparatus of claim 6 wherein said third tunnel is larger in circumference than said first and second tunnels.

8. The colon hydrotherapy apparatus of claim 6 wherein said third circular opening has a circumference equal to or larger than a circumference of said first and second circular openings.

9. The colon hydrotherapy device of claim 2 wherein said temperature manifold comprises a polymer block member having:
a first circular opening and a second circular opening within a firstside of said polymer block member;
a first tunnel and a second tunnel contiguous with said first and second circular openings, respectively, wherein a circumference of said first and second tunnels is smaller than a circumference of said first and second circular openings;
a third circular opening within a second side of said polymer block member; and
a third tunnel contiguous with the third circular opening wherein a circumference of said third tunnel is smaller than a circumference of said third circular opening and said first and second tunnels are contiguous with and open into said third tunnel.

10. The colon hydrotherapy apparatus of claim 9 wherein said third tunnel is equal in circumference to the circumference of said first and second tunnels.

11. The colon hydrotherapy apparatus of claim 9 wherein said third circular opening has a circumference equal to or larger than a circumference of said first and second circular openings.

12. A pressure manifold for a colon hydrotherapy device comprising a polymer block member having:
a first circular opening and a second circular opening within a first side of said polymer block member;
a first tunnel and a second tunnel contiguous with said first and second circular openings, respectively, wherein a circumference of said first and second tunnels is smaller than a circumference of said first and second circular openings;
a third circular opening within a second side of said polymer block member; and
a third tunnel contiguous with the third circular opening wherein a circumference of said third tunnel is equal to a circumference of said third circular opening and said first and second tunnels are contiguous with and open into said third tunnel.

13. The pressure manifold of claim 12 wherein said third tunnel is larger in circumference than said first and second tunnels.

14. The pressure manifold of claim 12 wherein said third circular opening has a circumference equal to or larger than a circumference of said first and second circular openings.

15. A temperature manifold for a colon hydrotherapy device comprising a polymer block member having:
a first circular opening and a second circular opening within a first side of said polymer block member;
a first tunnel and a second tunnel contiguous with said first and second circular openings, respectively, wherein a circumference of said first and second tunnels is smaller than a circumference of said first and second circular openings;
a third circular opening within a second side of said polymer block member; and
a third tunnel contiguous with the third circular opening wherein a circumference of said third tunnel is smaller than a circumference of said third circular opening and said first and second tunnels are contiguous with and open into said third tunnel.

16. The temperature manifold of claim 15 wherein said third tunnel is equal in circumference to the circumference of said first and second tunnels.

17. The temperature manifold of claim 15 wherein said third circular opening has a circumference equal to or larger than a circumference of said first and second circular openings.
